(19) United States
(12) Patent Application Publication
(10) Pub. No.: US 2008/0038317 A1
(43) Pub. Date: Feb. 14, 2008

(54) Therapeutic Lacrimal Canalicular Inserts And Related Methods

(76) Inventors: Chin-Ming Chang, Tustin, CA (US); Rhett M. Schiffman, Laguna Beach, CA (US); James N. Chang, Newport Beach, CA (US); R. Scott Jordan, Canyon, CA (US)

Correspondence Address: ALLERGAN, INC., 2525 DUPONT DRIVE, T2-7H, IRVINE, CA, 92612-1599, US

(21) Appl. No.: 11/572,097

(22) PCT Filed: Sep. 7, 2005
(86) PCT No.: PCT/US05/32222

§ 371 Date: Jun. 19, 2007

Related U.S. Application Data
(60) Provisional application No. 60/608,628, filed on Sep. 10, 2004.

(51) Int. Cl. A61F 2/14; A61K 38/12; A61P 27/00

(52) U.S. Cl. 424/428; 514/11

(57) ABSTRACT

Lacrimal canalicular inserts include a polymeric component and a therapeutic component. The therapeutic component is released from the inserts for extended periods of time, such as for more than about 2 weeks after placement in a lacrimal canaliculus of an individual. The polymeric component may include one or more non-biodegradable polymers, one or more biodegradable polymers, or combinations thereof. The therapeutic component may include one or more therapeutic agents. Therapeutically effective amounts of the therapeutic component are released from the insert and provide sustained drug delivery to the eye and/or the nasolacrimal system of the individual.

### CROSS-REFERENCE TO RELATED APPLICATIONS

This application claims the benefit of U.S. Application No. 60/608,628, filed Sep. 10, 2004, the entire contents of which are hereby incorporated by reference.

### BACKGROUND

The present invention relates to drug delivery systems. More particularly, the invention relates to sustained release lacrimal canalicular inserts which comprise one or more therapeutic agents. Such inserts are effective in providing extended or sustained release of the therapeutic agent or agents on or into the eye of an individual.

Treatment of ocular ailments and diseases may require topical application of medications to the eye. The most common topical dosage forms are solutions, suspensions, gels, and semisolids. These dosage forms suffer from short residence time at the absorption site due to consistent tear turnover and drainage. To overcome this shortcoming, frequent dosing is often required for topical ophthalmic dosage forms in order to achieve the desired disease management and treatment. This is inconvenient to patients, and poor compliance is a major reason for unsuccessful disease management and treatment for ocular diseases.

Some sustained release ocular drug delivery systems have been developed to assist with the treatment of ocular ailments and diseases. These sustained release drug delivery systems were developed with a goal of delivering therapeutic agents to the eye over a long period of time, often weeks to months, so the treatment wouldn't be compromised as a result of poor compliance. In many instances, however, such drug delivery systems have not been widely accepted due to their inherent discomfort (such as inserts for the cul-de-sac) or incapability of being manufactured at large scales.

One example of a dosage form is a biodegradable insert developed to be placed in the cul-de-sac of an eye to extend drug residence time and prolong drug absorption. Although this insert has been commercially available for years, it has not been popular due to inherent discomfort of the insert in the cul-de-sac and frequent falling off from the eye.

Another example dosage form, which is relatively more comfortable, is a punctum plug reservoir system. The use of this system for the sustained release of ophthalmic medications has been described in U.S. Pat. Nos. 6,196,933 and 3,949,750. In these plugs, a drug reservoir pore is designed within the plug to store and release medication onto the eye over time while the plug is positioned in the eyelid. These punctal plug reservoir systems suffer from large-scale manufacturing difficulties associated with reproducibly adding drugs into the reservoir of the plugs. In addition, the amount of drug that can be contained in the reservoir of the plug is limited by the size of the reservoir.

Some drug delivery devices and methods have been described. For example, U.S. Pat. No. 2,962,023 (Chappaz et al.) discloses a medicator. U.S. Pat. No. 3,710,795 (Higuchi et al.) discloses a drug-delivery device with stretched, rate controlling membrane. U.S. Pat. No. 3,832,252 (Higuchi et al.) discloses a method of making a drug-delivery device. U.S. Pat. No. 3,949,750 (Freeman) discloses a punctum plug and method for treating keratoconjunctivitis sicca (dry eye) and other ophthalmic ailments using same. U.S. Pat. No. 3,960,150 (Hussain et al.) discloses a biodegradable ocular device. U.S. Pat. No. 4,014,335 (Arnold) discloses an ocular drug delivery device. U.S. Pat. No. 4,131,648 (Choi et al.) discloses structured orthoester and orthocarbonate drug delivery devices. U.S. Pat. No. 4,249,531 (Heller et al.) discloses a biodegradable system for delivering drug manufactured from poly(carboxylic acid). U.S. Pat. No. 4,322,323 (Capozza) discloses an erodible device comprising surfactant for modifying the rate of erosion of the device. U.S. Pat. No. 4,660,546 (Herrick et al.) discloses a method for treating deficiency of tears. U.S. Pat. No. 4,851,228 (Zentner et al.) discloses a multi-particulate controlled porosity osmotic. U.S. Pat. No. 4,915,684 (MacKeen et al.) discloses a method and apparatus for modulating the flow of lacrimal fluid through a punctum and associated canaliculus. U.S. Pat. No. 5,163,959 (Herrick) discloses a method for treating an eye with a canalicular implant having a collapsible flared section. U.S. Pat. No. 5,545,208 (Wolff et al.) discloses an intraluminal drug eluting prosthesis. U.S. Pat. No. 5,575,815 (Slepian et al.) discloses a local polymeric gel therapy. U.S. Pat. No. 5,634,946 (Slepian) discloses a polymeric endoluminal paving process. U.S. Pat. No. 5,843,156 (Slepian et al.) discloses a local polymeric gel cellular therapy. U.S. Pat. No. 5,871,535 (Wolff et al.) discloses an intraluminal drug eluting prosthesis. U.S. Pat. No. 6,004,346 (Wolff et al.) discloses an intraluminal drug eluting prosthesis. U.S. Pat. No. 6,196,993 (Cohan et al.) discloses an ophthalmic insert and method for sustained release of medication to the eye. U.S. Pat. No. 6,290,684 (Herrick) discloses a punctum plug having a collapsible expanded section and distal tip extending substantially perpendicular thereto and method of inserting same. U.S. Pat. No. 6,344,047 (Price et al.) discloses an instrument for inserting a punctum plug and method for manufacturing the instrument. U.S. Pat. No. 6,706,034 (Bhat) discloses a process for agent retention in biological tissues. U.S. Patent Publication No. 2002/0138154 (Li et al.) discloses controlling resorption of bioresorbable medical implant material. U.S. Patent Publication No. 2003/0097151 (Smedley et al.) discloses an apparatus and mitochondrial treatment for glaucoma. U.S. Patent Publication No. 2004/0102729 (Haffner et al.) discloses devices and methods for glaucoma treatment.

U.S. Pat. No. 6,713,081 discloses ocular implant devices made from polyvinyl alcohol and used for the delivery of a therapeutic agent to an eye in a controlled and sustained manner. The implants may be placed subconjunctivally or intravitreally in an eye.

Biocompatible implants for placement in the eye have also been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; and 6,699,493.

An ophthalmic dosage form which can resolve compliance issues, which is easy to administer to a patient, and easy to manufacture is highly desired. Thus, there is a need for new drug delivery systems for providing desired therapeutic effects of ophthalmic conditions of eyes of humans or animals.

### SUMMARY

New systems and methods for treating ophthalmic conditions of eyes of humans or animals are provided. The present drug delivery systems are highly suitable for extended delivery of one or more therapeutic agents to an eye and/or nasolacrimal system and provide therapeutic effects to the eye, which may be effective in stabilizing, enhancing or improving a patient's vision. The present drug delivery systems are structured to be fixedly placed in a lacrimal canaliculus of an individual. The present drug delivery systems are relatively easy to manufacture, compared to previously described punctal plugs, address patient compliance concerns of administering therapeutic agents to an eye, and provide enhancements in the amount of therapeutic agent that may be provided in the drug delivery systems.

In one embodiment of the present invention, a lacrimal canalicular insert, comprises a matrix of a polymeric component and a therapeutic component. The therapeutic component is distributed substantially throughout the matrix.

In another embodiment, a biodegradable lacrimal canalicular insert comprises a biodegradable polymer component and a therapeutic component in a member. The member is structured to be placed in a lacrimal canaliculus of an individual and to release the therapeutic component onto an eye of the individual.

In one specific embodiment, a biodegradable lacrimal canalicular insert comprises an extrusion molded member comprising a blend of at least one biodegradable polymer and at least one therapeutic agent.

The polymers of the present inserts may be any polymeric material that is biologically inert, and non-allergenic.

In another aspect of the present invention, a method of producing a lacrimal canalicular insert, comprises forming at least one biodegradable polymer and at least one therapeutic agent into a member structured to be placed in a lacrimal canaliculus of an individual.

In yet another aspect, a method of treating a condition of an eye of a human or animal comprises placing an insert, as described herein, into a lacrimal canaliculus of the human or animal.

The polymers of biodegradable inserts should degrade or erode for extended periods of time. The erosion of the polymers sustains the drug release from the insert.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

These and other aspects and advantages of the present invention are apparent in the following detailed description, drawings, and claims.

### BRIEF DESCRIPTION OF THE DRAWINGS

FIG. 1 is an illustration of a sectional view of an lacrimal canalicular insert in accordance with the disclosure herein.

FIG. 2 is an illustration of the insert of FIG. 1 placed in a lacrimal canaliculus of an individual.

FIG. 3 is an illustration of the insert of FIG. 1 including a coating.

FIG. 4 is an illustration of the insert of FIG. 3 including an axial bore.

FIG. 5 is a top plan view along line 5-5 of FIG. 4.

### DETAILED DESCRIPTION

As understood by persons of ordinary skill in the art, tears are produced by the lacrimal gland superior to the outer portion of each eye of an individual, such as a human. Tears flow across the surface of the eye to a shallow pool, termed the lacrimal lake, which is located where the eyelids come together at their inner ends. From there, the tears drain through small openings in each of the eyelids, namely the upper lacrimal punctum and the lower lacrimal punctum. The lacrimal puncta may also be understood to be lacrimal punctal apertures. From the upper and lower puncta, the tears pass into the upper lacrimal canaliculus and lower lacrimal canaliculus, respectively. These lacrimal canaliculae are duct-like pathways leading to the lacrimal sac. The lacrimal canaliculus may also be referred to as a lacrimal canal or lacrimal canaliculus. The lacrimal sac is the superior, expanded portion of the nasolacrimal duct which drains tears into the nasal system. The upper lacrimal punctum and upper lacrimal canaliculus are said to drain about 10% of the tears from the eye, such that their obstruction virtually never leads to tear overflow.

Insufficient tears, or “dry eye”, is a common condition caused by insufficient production of tears from the lacrimal gland which causes symptoms such as dryness, redness, burning, reflex tearing, itching, or foreign body sensation. In especially difficult cases of dry eye, a punctal occluder or punctal plug may be placed into one or both of the lacrimal puncta. Punctal occluders prevent the tears, which are being produced in deficient volume by the lacrimal gland, from draining into the lacrimal canaliculae. Punctal occluders can be secured in the lacrimal puncta without anesthesia and removed with ease when necessary.

Existing punctal occluders typically include a collarette or head portion which rests on the exterior of the punctum, and a body portion that projects into the lacrimal canaliculus. The body portion typically includes a bulb that blockingly projects into the canaliculus, and a neck connecting the collarette and the bulb. Commercially available punctal occluders usually have a length of approximately 2.0 mm, and differ from each other only slightly in configuration. The bulbs of the punctal occluders are designed to prevent the occluder from being easily dislodged from the canaliculus, and may be tapered for ease of insertion into the puncta. The collarettes are designed to have a diameter to prevent the occluder from completely entering the canaliculus and are preferably smooth to minimize irritation of the eye. The necks of different punctal occluders are similar and essentially a non-functional connection between the collarette and the bulb portions. The collarette may include an aperture extending into the neck to aid in grasping the occluder during its insertion into the puncta.

Examples of punctal occluders can be found in U.S. Pat. Nos. 3,949,750 and 5,283,063 issued to Freeman, U.S. Pat. Nos. 5,053,030; 5,171,270; and 5,723,005 issued to Herrick, U.S. Pat. No. 5,417,651 issued to Guena et al., U.S. Pat. No. 5,423,777 issued to Tajiri et al., and U.S. Pat. No. 6,196,993 issued to Cohan et al. Punctal occluders which are used to reduce or prevent tear drainage are typically made of non-biodegradable materials. In U.S. Pat. No. 6,196,993, the punctal occluder comprises a non-biodegradable shell encapsulating a reservoir containing medication. Or, stated differently, the punctal occluder disclosed in U.S. Pat. No. 6,196,993 includes two discrete regions, one region containing a medication, and a second region devoid of medication.

The present invention involves drug delivery devices or systems, such as lacrimal canalicular inserts, that provide therapy to a patient. In accordance with the disclosure herein, biodegradable or non-biodegradable inserts are disclosed that are useful for placement into a lacrimal canaliculus of a human or animal, and preferably a living human or animal. Such inserts are preferably administered into a lacrimal canaliculus of a patient to provide extended or sustained release of one or more therapeutic agents to the individual's eye and/or nasolacrimal system and/or nose. The insert is effective in providing prolonged delivery of one or more therapeutic agents to the eye, for example, to the outer surface of the eye. The insert is structured, such as size and/or shaped, to be retained in lacrimal canaliculus while the therapeutic agent is being released. The therapeutic agent or agents may then be delivered to the interior of the eye to provide a desired therapeutic effect, such as the treatment of an ophthalmic condition, or one or more symptoms thereof. The therapeutic agent or agents may provide therapeutic effects to conditions affecting one or more portions of the eye, for example, an anterior portion of the eye, a posterior portion of the eye, or a combination thereof.

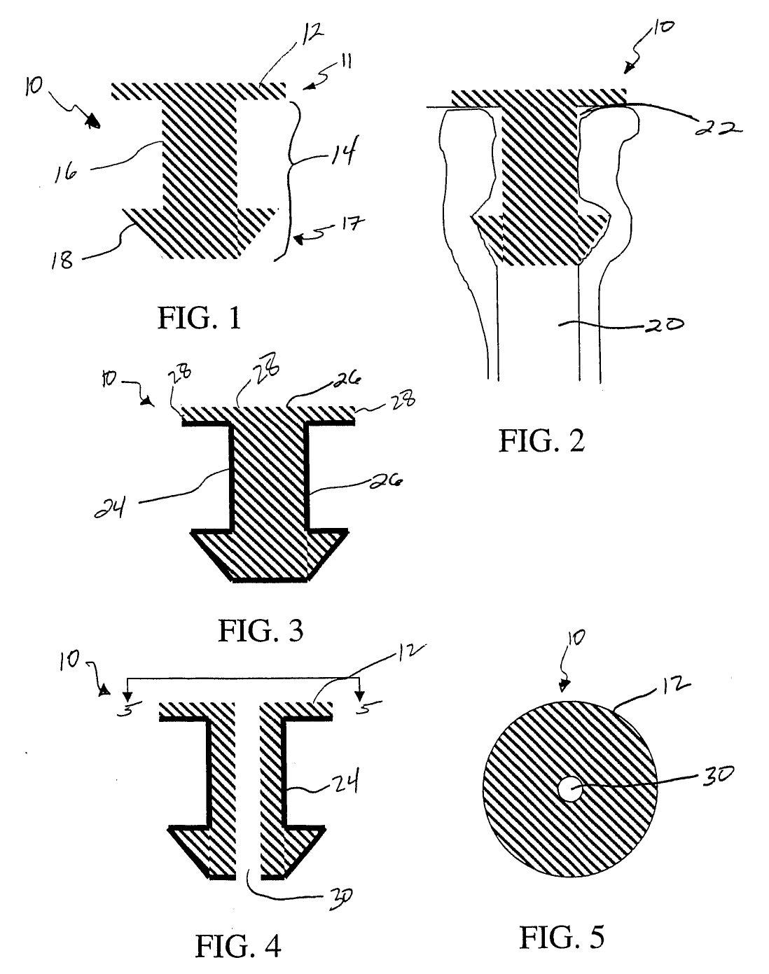

One example of a lacrimal canalicular insert 10 is illustrated in FIG. 1. Lacrimal canalicular insert 10 comprises a head portion 12 and a body portion 14. The head portion 12 is structured, such as sized and/or shaped, to be placed in proximity to a punctum of an individual in need of therapy, the body portion 14 is structured to be placed in a lacrimal canaliculus of the individual. The body portion 14 comprises a neck 16 and a barb 18. The insert 10 may also be understood to comprise a proximal end 11 and a distal end 17. The proximal end 11 comprises the head portion 12, and the distal end 17 comprises the barb 18. As shown in FIG. 1, the neck 16 is located between the distal end 17 and the head portion 12, and the distal end has a greater diameter relative to the neck 16. Other configurations and structures may be used for the inserts so long as the inserts are structured to be retained in a lacrimal canaliculus.

FIG. 2 illustrates the insert 10 located in a lacrimal canaliculus 20 of an individual. The body portion 14 is located in the lacrimal canaliculus 20, and the head portion 12 is located in proximity to the punctum 22. The insert 10 may be placed in either an upper or lower lacrimal canaliculus. In certain embodiments, the insert 10 is placed in the upper lacrimal canaliculus to maintain sufficient amounts of tear drainage. In other embodiments, the insert 10 is structured to be placed in the lower canaliculus. Such an insert may effectively deliver one or more therapeutic agents to the eye and/or nasolacrimal system and/or nose, and reduce tear flow or drainage from the eye.

As shown in FIG. 2, the insert 10 may be understood to be a punctal plug or punctal occluder. Accordingly, the head portion 12 is structured to block or occlude the punctal opening 22 of the lacrimal canaliculus. The barb 18 is structured to engage with the wall of the lacrimal canaliculus 20 and is effective to reduce the likelihood that the insert 10 will be inadvertently dislodged from the lacrimal canaliculus 20. Other embodiments of the present inserts may not occlude the punctum 22. For example, the insert may be structured to be placed inside a lacrimal canaliculus away from the punctal aperture 22. In situations in which the insert is placed in a lower lacrimal canaliculus, tear drainage may still occur albeit at a reduced rate relative to a lacrimal canaliculus without an insert, and the therapeutic agents may be released from the insert.

The insert 10 is structured to be easily inserted into the lacrimal canaliculus 20 and does not cause substantial irritation to the eye or the individual in which the insert 10 is placed. The insert comprises substantially smooth surfaces and is made of materials that are biocompatible, for example ophthalmically acceptable.

FIG. 3 illustrates the insert 10 with a coating 24. The coating 24 is provided over a major portion of the insert 10. As shown in FIG. 3, the insert 10 has a peripheral surface 26. The peripheral surface 26 includes a head portion peripheral surface 28. The coating 24 is illustrated as covering the peripheral surface 26 of the insert 10 except for the head portion peripheral surface 28, or the portions of the peripheral surface 26 which contact an eye of the individual. The coating 24 in the illustrated embodiment is substantially impermeable to a therapeutic component of the insert, as discussed herein. In certain embodiments, the coating 24 comprises a non-biodegradable polymer.

As shown in FIG. 4, the insert 10 comprises an aperture 30 in the coating 24 provided at the distal end of the insert. In the illustrated embodiment, the aperture 30 extends through the insert 10 to the head portion 12. Thus, the insert 10 illustrated in FIGS. 4 and 5 may be understood to comprise an axial bore extending the length of the insert 10.

The insert 10 comprises a polymeric component and a therapeutic component. The polymeric component and the therapeutic component are associated, such as blended or mixed, with each other to provide extended release of the therapeutic component from the insert when the insert is placed in a lacrimal canaliculus. The insert 10 may be structured so that the therapeutic component is released from the insert for at least about one month after the insert is placed in the lacrimal canaliculus. In certain embodiments, the insert 10 may release the therapeutic component for more than one month, such as for at least about three months, at least about six months, at least about twelve months, or even longer. Furthermore, certain of the present inserts may release the therapeutic component for at least about two weeks. The present inserts are structured to release the therapeutic component for extended periods of time relative to conventional topical administration methods of therapeutic agents.

In certain embodiments of the present inserts, the lacrimal canalicular insert comprises, consists essentially of, or consist entirely of, a matrix of a polymeric component and a therapeutic component. The therapeutic component of such inserts is distributed substantially throughout the matrix. As shown in FIG. 1, the matrix may be structured, such as sized and/or shaped, in the form of a punctal plug or punctal occluder.

The polymeric component of the foregoing inserts may comprise one or more non-biodegradable polymers, one or more biodegradable polymers, or mixtures thereof. The present non-biodegradable insert may release the therapeutic agent or agents by a diffusion-like process where fluids, such as tear fluid, wet the therapeutic component and allows the therapeutic component to pass or be released from the insert.

As shown in FIG. 3, the insert may comprise a non-biodegradable coating around a major portion of the matrix.

When the insert comprises a non-biodegradable polymer matrix and a therapeutic component, the insert may be structured to occlude the lacrimal canaliculus in which the insert is placed after the therapeutic component has been released therefrom. For example, such an insert comprising a therapeutic component may be placed in a lower lacrimal canaliculus to provide drug delivery to the eye or to the nasolacrimal system of an individual. After the therapeutic component has been substantially depleted, the insert may be left in place in the lacrimal canaliculus to block tear drainage from the eye, thereby occluding the lacrimal canaliculus. Thus, the insert may be used in combination therapies, such as a drug delivery system and a punctal occluder.

When the foregoing inserts comprise one or more biodegradable polymers, such as the biodegradable polymers disclosed herein, the insert may not need to be removed since the insert will have degraded and have been absorbed by or flushed from the individual's body.

In other embodiments of the present inserts, the lacrimal intracanalicular insert comprises, consists essentially of, or consists of, a biodegradable polymer component and a therapeutic component structured to be placed in a lacrimal canaliculus. Such inserts may or may not be provided in the form of a matrix. However, such inserts are structured to release the therapeutic component therefrom for extended periods of time, as discussed herein.

A biodegradable insert is biodegraded and/or bioeroded when placed in a lacrimal canaliculus and as the insert is releasing the therapeutic agent or agents. The components of the insert may be absorbed by the patient's body thereby reducing, and preferably eliminating, the need to surgically remove the insert after the therapeutic agent or agents have been released.

The term “biodegradable polymer component” refers to a portion of the insert which comprises one or more polymers that degrade or erode in vivo, and wherein erosion of the polymer or polymers over time occurs concurrent with or subsequent to release of the therapeutic component. The terms “biodegradable” and “bioerodible” are equivalent and are used interchangeably herein.

The polymeric component of the present inserts, including the biodegradable polymer, may be a homopolymer, a copolymer, or a polymer comprising more than two different polymeric units. The polymer or polymers may be cross-linked together, or may be associated with each other in a mixture, blend, matrix or network of polymers.

The present biodegradable inserts release the therapeutic component by being eroded or degraded, and not necessarily by diffusion. Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both.

The biodegradable polymer component may comprise, consist essentially of, or consist of one or more biodegradable synthetic polymers. In certain implants, the biodegradable polymer component comprises at least two different biodegradable polymers. In other implants, the biodegradable polymer component comprises at least one biodegradable copolymer.

Suitable polymeric materials or compositions of the present inserts include those materials which are compatible, that is biocompatible, with the eye so as to cause no substantial interference with the functioning or physiology of the eye. Such materials may be at least partially and more preferably substantially completely biodegradable or bioerodible.

The biodegradable polymeric component is provided in an amount in the insert that is effective in delaying release of the therapeutic component after the insert is placed in the lacrimal canaliculus.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

In certain inserts, the biodegradable polymeric component is selected from the group consisting of polylactides, polyglycolides, polycaprolactones, polyanhydrides, polyamides, polyurethanes, polyesteramides, polyorthoesters, polydioxanones, polyacetals, polyketals, polycarbonates, polyorthoesters, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), collagen, chitin, chitosan, and copolymers, terpolymers, derivatives thereof and mixtures thereof.

For example, the biodegradable polymeric component of the present inserts may be selected from the group consisting of poly lactic acid, poly glycolic acid, poly lactic acid/glycolic acid (PLGA), derivatives thereof, and mixtures thereof.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

The biodegradable polymeric materials which are included to form the insert are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implant. Different molecular weights of the same or different polymeric compositions may be included in the implant to modulate the release profile

In some implants, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the implant, where a more flexible implant is desirable for larger geometries. The % of polylactic acid in the polylactic acid polyglycolic acid (PLGA) copolymer can be 0-100%, preferably about 15-85%, more preferably about 35-65%. In some implants, a 50/50 PLGA copolymer is used.

As discussed herein, the present inserts may release the therapeutic component for more than two weeks after implantation into a lacrimal canaliculus. In comparison, temporary punctal plugs which have been identified in patents and do not include a therapeutic component, degrade or are removed within about 2 weeks of placement in a lacrimal canaliculus. In certain of the present inserts, therapeutically effective amounts of the therapeutic component are released for more than about one month, and even for about six months or more, as discussed herein.

As used herein, a “therapeutic component” refers to a portion of the insert, which comprises one or more therapeutic agents or substances used to treat a medical ophthalmic disease or condition of the eye and/or to otherwise beneficially affect a patient's vision. The therapeutic component may be provided in a discrete region of insert, such as in the biodegradable inserts, or the therapeutic component may be homogenously distributed throughout the insert, such as inserts comprising a matrix of a non-biodegradable polymer, a biodegradable polymer, or combinations thereof. The therapeutic agents of the therapeutic component are typically ophthalmically acceptable, and are provided in a form that does not cause adverse reactions when administered to an eye. The therapeutic agents may also be therapeutically effective in treating or relieving one or more conditions of the nasolacrimal system.

In certain embodiments of the present implants, the therapeutic component comprises two or more different therapeutic agents.

The therapeutic component of the present inserts may comprise one or more therapeutic agents selected from the group consisting of steroidal anti-inflammatory agents, non-steroidal anti-inflammatory agents, retinoids, prostaglandins, tyrosine kinase inhibitors, adrenoreceptor agonists, adrenoreceptor antagonists, dopaminergic agonists, cholinergic agonists, carbonic anhydrase inhibitors, guanylate cyclase activators, cannabinoids, endothelin, adenosine agonists, antianagiogenic compounds, angiostatic compounds, neuroprotectants, analgesics, antipyretics; antihistamines, antibiotics, beta blockers, anti-neoplastic agents, immunosupressive agents, antiviral agents, antioxidants, and mixtures thereof.

Examples of steroidal anti-inflammatory agents include corticosteroids. In view of the above, the inserts may comprise a corticosteroid component. For example, the corticosteroid component may comprise one or more corticosteroids. The corticosteroid component is provided in a therapeutically effective amount, such as an amount which is effective in providing a therapeutic effect when the corticosteroid component is released from the insert to the eye.

The corticosteroid component may include without limitation, one or more corticosteroids selected from the group consisting of alclometasone dipropionate, amcinonide, amcinafel, amcinafide, beclamethasone, betamethasone, betamethasone dipropionate, betamethasone valerate, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone, diflorasone diacetate, dichlorisone, esters of betamethasone, fluazacort, flucetonide, flucloronide, fludrotisone, fluorocortisone, flumethasone, flunisolide, fluocinonide, fluocinolone, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, loteprendol, medrysone, meprednisone, methylprednisone, methylprednisolone, mometasone furoate, paramethasone, paramethasone acetate, prednisone, prednisolone, prednisolone acetate, prednidone, triamcinolone acetonide, triamcinolone hexacatonide, and triamcinolone, salts thereof, derivatives thereof, and mixtures thereof. In one embodiment of the present inserts, the corticosteroid component comprises, consists essentially of, or consists only of triamcinolone acetonide.

As used herein, the term “derivative” refers to any substance which is sufficiently structurally similar to the material which it is identified as a derivative so as to have substantially similar functionality or activity, for example, therapeutic effectiveness, as the material when the substance is used in place of the material. The functionality of any derivative disclosed herein may be determined using conventional routine methods well known to persons of ordinary skill in the art.

Other steroids which may be useful in the present compositions include, without limitation, glucocorticoids, androgenic steroids, estrogenic steroids, and non-estrogenic steroids.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of antineoplastic agents include adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppresive agents include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Other therapeutic agents include squalamine, carbonic anhydrase inhibitors, alpha agonists, prostamides, prostaglandins, antiparasitics, antifungals, tazarotene, tazarotenic acid, and derivatives thereof.

Some additional specific therapeutic agents include brimonidine, timolol, bimatoprost, latanoprost, travoprost, unoprostone isopropyl, cyclosporine, memantine, salts thereof, and mixtures thereof.

The therapeutic agent or agents of the present inserts may include any and all salts, and prodrugs or precursors of the therapeutic agents, including those specifically identified herein.

The amount of therapeutic agents employed in the insert, individually or in combination, will vary widely depending on the effective dosage required and the desired rate of release from the insert. As indicated herein, the agent will be at least about 1, more usually at least about 10 weight percent of the insert, and usually not more than about 80, more usually not more than about 60 weight percent of the insert. For example, the therapeutic component may be provided in an amount from about 20% to about 80% of the weight of the insert, for example from about 40% to about 60% of the weight of the insert. In certain inserts, the therapeutic component is about 50% of the weight of the insert.

In addition to the therapeutic component, the present inserts may comprise effective amounts of one or more agents selected from the group consisting of solublilizers, plasticizers, stabilizers, buffers, salts, preservatives, and the like.

Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total insert.

Suitable water soluble preservatives include chlorite components, such as stabilized chlorine dioxide, metal chlorites, and the like, sodium bisulfite, sodium bisulfate, sodium thiosulfate, ascorbate, benzalkonium chloride, chlorobutanol, thimerosal, hexetidine, phenylmercuric acetate, phenylmercuric borate, phenylmercuric nitrate, parabens, methylparaben, ethylparaben polyvinyl alcohol, benzyl alcohol, phenylethanol and the like and mixtures thereof. These agents may be present in amounts of from 0.001 to about 5% by weight and preferably 0.01 to about 2% by weight.

Examples of salts include, without limitation, sodium chloride and potassium chloride.

In addition, the inserts may include a solubility enhancing component provided in an amount effective to enhance the solubility of the therapeutic component relative to substantially identical inserts without the solubility enhancing component. For example, an insert may include a cyclodextrin. Examples of useful cyclodextrins include, but are not limited to: α-cyclodextrin, derivatives of α-cyclodextrin, β-cyclodextrin, derivatives of β-cyclodextrin, γ-cyclodextrin, derivatives of γ-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-β-cyclodextrin, sulfobutylether-β-cyclodextrin, and the like and mixtures thereof. As used herein, the term “derivatives” as it relates to a cyclodextrin means any substituted or otherwise modified compound which has the characteristic chemical structure of a cyclodextrin sufficiently to function as a cyclodextrin component, for example, to enhance the solubility and/or stability of active components and/or reduce unwanted side effects of the active components and/or to form inclusive complexes with active components, as described herein. The cyclodextrin may be provided in an amount from about 0.5% (w/w) to about 25% (w/w) of the insert. In certain implants, the cyclodextrin is provided in an amount from about 5% (w/w) to about 15% (w/w) of the insert. Inserts which comprise a solubility enhancing component may be effective in delivering therapeutically effective amounts of the therapeutic agent or agents to the interior of the eye, for example the posterior or posterior portion, of the eye. Thus, in one embodiment, a lacrimal canalicular insert may comprise, or consist essentially of, a solubility enhancing component and a therapeutic component. For example, such an insert may comprise a cyclodextrin and one or more therapeutic agents, including each and every therapeutic agent disclosed herein. Such an insert may comprise a polymeric component, including without limitation, the non-biodegradable and biodegradable polymers disclosed herein.

The present inserts may also include one or more release modulators, such as one or more agents that are effective in controlling the release rate of the therapeutic component from the insert. Examples of useful release modulators include those described in U.S. Pat. No. 5,869,079. The release modulator may accelerate the release of the therapeutic component or decelerate the release of the therapeutic component. For example, the release modulator may increase or decrease the erosion rate of biodegradable implants, or the diffusion rate in non-biodegradable implants. Thus, it is possible to provide pulsatile or continuous or substantially constant release profiles of the therapeutic component to the eye.

The present inserts may be understood to comprise a member which comprises a polymeric component, such as a biodegradable polymer component, and a therapeutic component. The member of the present inserts, including the biodegradable inserts, may also comprise a structural enhancement component effective in maintaining the strength and physical structure of the member. The structural enhancement component may comprise a non-biodegradable polymer mixed with the biodegradable polymer component and therapeutic component.

The present inserts may also comprise one or more pore-forming agents. Pores may be formed in the insert by incorporating water-soluble materials into the polymer component. Examples of pore-forming agents include sugars, salts, and polymers, such as polymers that are not soluble in the biodegradable polymeric component or its carrier solvent. For example, the pore-forming agents may include one or more of sucrose, dextrose, sodium chloride, sodium carbonate, hydroxypropylcellulose, carboxymethylcellulose, polyethylene glycol, and polyvinylpyrollidone. The pores may have a diameter from about 3 μm to about 500 μm. For example, the pores may have a diameter from about 10 μm to about 250 μm. The therapeutic component may also act as a pore forming agent for the insert.

Advantageously, by providing the therapeutic component in a matrix of a non-biodegradable polymer or a biodegradable polymer, or combinations thereof, or by providing the therapeutic component with a biodegradable polymer component, the release of the therapeutic component can be controlled to a greater degree than other inserts which deliver therapeutic agents. For example, the release of the therapeutic component may include an initial burst of release followed by a gradual increase in the amount of the therapeutic component released, or the release may include an initial delay in release of the therapeutic component followed by an increase in release. When the insert is substantially completely degraded, the percent of the therapeutic component that has been released is about one hundred.

In addition, the therapeutic component may be released at a relatively constant rate over the life of the insert. For example, the therapeutic component may be released at a substantially constant rate of about 0.01 μg to about 2 μg per day. Thus, the release rate profile of such an insert may be linear.

In other inserts, the release rate profile may be non-linear. In other words, the therapeutic component may be released at two or more different rates over the life of the insert.

As described herein, certain of the present inserts are monolithic. For example, the insert 10 illustrated in FIG. 1 comprises a matrix of a therapeutic component and a polymeric component. In comparison, previously disclosed inserts include an encapsulated reservoir of a medication. Monolithic inserts provide advantages such as ease of manufacture and controllability of release rates.

In accordance with the disclosure herein, certain embodiments of a lacrimal canalicular insert may comprise a biodegradable portion and a non-biodegradable portion, wherein the biodegradable portion comprises a therapeutic component. The therapeutic component may be released from the insert by bioerosion, as discussed herein, and the insert may be effective as a punctal occluder when the biodegradable portion has been degraded.

Various techniques may be employed to produce the present inserts described herein. Useful techniques include, but are not necessarily limited to, solvent evaporation methods, phase separation methods, liquid absorption methods, interfacial methods, molding methods, injection molding methods, extrusion methods, co-extrusion methods, carver press methods, die cutting methods, heat compression, combinations thereof and the like.

In the illustrated embodiment of the present inserts, extrusion methods are used in the manufacture of the inserts. Thus, in accordance with the disclosure herein, the insert may comprise a matrix of an extrusion molded blend of the polymeric component and the therapeutic component. In other inserts, such as the biodegradable inserts, the biodegradable polymer component and the therapeutic component are present in an extrusion molded member.

Accordingly, in one embodiment of the present inserts, a biodegradable lacrimal canalicular insert comprises an extrusion molded blend of at least one biodegradable polymer and at least one therapeutic agent.

In another aspect of the present invention, a method of producing a lacrimal canalicular insert comprises forming at least one biodegradable polymer and at least one therapeutic agent into a member structured to be placed in a lacrimal canaliculus of an individual. The forming may comprise extrusion molding a mixture of the biodegradable polymer and the therapeutic agent.

The method may also comprise an optional step of applying a coating to a peripheral surface of the member.

The method may also comprise forming an axial bore in the member.

Extrusion methods for non-lacrimal canalicular drug delivery systems are known. Extrusion methods are advantageous due to a reduced need to use solvents in the manufacturing of the present inserts. When using extrusion methods, the polymer and therapeutic agent are chosen so as to be stable at the temperatures required for manufacturing. The extrusion temperature is usually set at the level where polymers are softened, deformed or melted. The incorporated therapeutic agent or drug may be dispersed or dissolved or molten in the drug/polymer mixture to be extruded. Extrusion methods use temperatures of about 25 degrees C. to about 150 degrees C., more preferably about 65 degrees C. to about 130 degrees C. For example, the extrusion temperature may be about 70 degrees Celsius. An implant may be produced by bringing the temperature to about 60 degrees C. to about 150 degrees C. for drug/polymer mixing, such as about 130 degrees C., for a time period of about 0 to 1 hour, 0 to 30 minutes, or 5-15 minutes. For example, a time period may be about 10 minutes, preferably about 0 to 5 min. The implants are then extruded at a temperature of about 60 degrees C. to about 130 degrees C., such as about 75 degrees C. Preferably, the therapeutic agent is stable at the extrusion temperature.

When a coating is to be provided on an insert, the insert may be co-extruded with a coating material.

In another embodiment, a composition comprising one or more polymers may be extruded and formed into lacrimal canalicular inserts. The extruded structure may then be immersed or otherwise contact a liquid composition comprising one or more therapeutic agents. The therapeutic agents may thus be incorporated into the extruded structure without actually extruding the therapeutic agents. A coating, as described herein, may then be provided over the insert.

In a further embodiment, a lacrimal canalicular insert may be formed by extruding a blend of a polymeric component and a therapeutic component, as described herein. The extruded member may then be immersed or otherwise placed in contact with a liquid composition comprising one or more of the same or different therapeutic agents. In certain situations, it may be desirable only to contact the head portion of the insert with the liquid composition. Thus, the insert may comprise an extruded molded blend of a polymeric component and a therapeutic component, and may further comprise a portion which has a relatively greater amount of a therapeutic component compared to the other portions of the insert. Such an insert may be useful in providing a rapid release of a relatively large amount of the therapeutic component followed by a sustained release of a relatively smaller, but therapeutic, amount of the therapeutic component to the patient.

The present inserts may be, and are preferably, sterile, for example, prior to being placed in a lacrimal canaliculus.

The present inserts may be placed in a lacrimal canaliculus of a patient to provide a treatment to a patient. For example, the composition may be administered to a human or animal patient to treat an ocular condition or disease.

Among the diseases/conditions which can be treated or addressed in accordance with the present invention include, without limitation, the following:

Anterior Segment Diseases: dry eye, Anterior uveitis, Conjuctivitis, Glaucomas, Keratitis, Lid diseases, Scleritis and episcleritis.

MACULOPATHIES/RETINAL DEGENERATION: Non-Exudative Age Related Macular Degeneration (ARMD), Exudative Age Related Macular Degeneration (ARMD), wet macular degeneration, Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis and the like.

The present inserts may be placed into a lacrimal canaliculus of an eye using any conventional technique. For example, the inserts may be placed in a lacrimal canaliculus using forceps, tweazers, or other similar instrument. In addition, the inserts may be placed in a lacrimal canaliculus using a needle, or other similar type of device which can penetrate the insert along its longitudinal axis and be removed therefrom when the insert engages a lacrimal canalicular wall. One example of an instrument suitable for inserting the present inserts is disclosed in U.S. Pat. No. 6,344,047.

In one embodiment of the present invention, an insert, such as the inserts disclosed herein, is placed in a lacrimal canaliculus at a location effective in releasing the therapeutic component from the insert for at least about one month after placement therein.

The method of the present invention may also comprise one or more additional steps of administering at least one other therapeutic agent to the eye of the human or animal. Such administration may comprise topical administration. It can be understood that the present inserts can be used in combination therapies by delivering one or more therapeutic agents for extended periods of time from placement in lacrimal canaliculus, and topically applying one or more therapeutic agents to the eye.

In addition, the present methods of treating an ophthalmic condition or disease of an eye may include administering a therapeutic agent via a lacrimal canalicular insert, and administering a solubility enhancing component to the eye. The solubility enhancing component, such as cyclodextrin, may be provided in the insert so that the combination of therapeutic agent or agents, and the solubility enhancing component are administered to the eye from the insert. Alternatively, or in addition, a solubility enhancing component may be administered separately from the insert, such as by topically administering the solubility enhancing component as a liquid, gel, ointment, and the like.

For example, an insert may be provided which comprises a therapeutic agent, and a cyclodextrin. The therapeutic agent and the cyclodextrin may be released from the insert and administered to the eye. The administration may be effective in treating a disease in the posterior of the eye. Such inserts may be effective in administering a greater amount of the therapeutic agent to the posterior of the eye relative to substantially identical inserts which comprise no cyclodextrin.

As another example, an insert comprising one or more therapeutic agents may be placed in a lacrimal canaliculus of an individual, such as a mammal. The therapeutic agent or agents may be released from the insert and delivered to the eye. A solubility enhancing component may be separately topically applied to the eye in the form of drops. The administration of drops of the solubility enhancing component may be effective in delivering the therapeutic agent into the eye, such as the posterior of the eye.

The following examples serve to illustrate certain preferred embodiments and aspects of the invention and are not to be construed as limiting the scope thereof.

### EXAMPLE 1

A lacrimal canalicular insert is made with dexamethasone and a polylactic acid/polyglycolic acid (PLGA) copolymer. 500 micrograms of dexamethasone powder and 500 micrograms of PLGA powder is mixed to form a mixture. The mixture is placed into an extruder, and is heated for 1 hour at a temperature between about 80° C. and about 110° C., such as about 95° C. The mixture is extruded into a mold having a desired shape for a lacrimal canalicular insert. The lacrimal canalicular insert has about 50% of the dexamethasone and 50% of the biodegradable polymer. The insert is stored in a sterile environment.

### EXAMPLE 2

A lacrimal canalicular insert is made as described in Example 1, except 250 micrograms of dexamethasone powder is mixed with 750 micrograms of PLGA powder to form an insert with a weight ratio of therapeutic component to polymeric component of 25:75.

### EXAMPLE 3

A lacrimal canalicular insert is made as described in Example 1, except 750 micrograms of dexamethasone powder is mixed with 250 micrograms of PLGA powder to form an insert with a weight ratio of therapeutic component to polymeric component of 75:25.

### EXAMPLE 4

A lacrimal canalicular insert is made as described in Example 1, except triamcinolone acetate powder is used instead of dexamethasone.

### EXAMPLE 5

A lacrimal canalicular insert is made as described in Example 2, except triamcinolone acetate powder is used instead of dexamethasone.

### EXAMPLE 6

A lacrimal canalicular insert is made as described in Example 3, except triamcinolone acetate powder is used instead of dexamethasone.

### EXAMPLE 7

A lacrimal canalicular insert is made as described in Example 1, except nepafenac powder is used instead of dexamethasone.

### EXAMPLE 8

A lacrimal canalicular insert is made as described in Example 2, except nepafenac powder is used instead of dexamethasone.

### EXAMPLE 9

A lacrimal canalicular insert is made as described in Example 3, except nepafenac powder is used instead of dexamethasone.

### EXAMPLE 10

A lacrimal canalicular insert is made as described in Example 1, except brimonidine powder is used instead of dexamethasone.

### EXAMPLE 11

A lacrimal canalicular insert is made as described in Example 2, except brimonidine powder is used instead of dexamethasone.

### EXAMPLE 12

A lacrimal canalicular insert is made as described in Example 3, except brimonidine powder is used instead of dexamethasone.

### EXAMPLE 13

A lacrimal canalicular insert is made as described in Example 1, except a combination of brimonidine and timolol is used instead of dexamethasone.

### EXAMPLE 14

A lacrimal canalicular insert is made as described in Example 2, except a combination of brimonidine and timolol is used instead of dexamethasone.

### EXAMPLE 15

A lacrimal canalicular insert is made as described in Example 3, except a combination of brimonidine and timolol is used instead of dexamethasone.

### EXAMPLE 16

A lacrimal canalicular insert is made as described in Example 1, except cyclosporine is used instead of dexamethasone.

### EXAMPLE 17

A lacrimal canalicular insert is made as described in Example 2, except cyclosporine is used instead of dexamethasone.

### EXAMPLE 18

A lacrimal canalicular insert is made as described in Example 3, except cyclosporine is used instead of dexamethasone.

### EXAMPLE 19

A lacrimal canalicular insert is made as described in Example 1, except bimatoprost is used instead of dexamethasone.

### EXAMPLE 20

A lacrimal canalicular insert is made as described in Example 2, except bimatoprost is used instead of dexamethasone.

### EXAMPLE 21

A lacrimal canalicular insert is made as described in Example 3, except bimatoprost is used instead of dexamethasone.

### EXAMPLE 22

A lacrimal canalicular insert is made as described in Example 1, except timolol is used instead of dexamethasone.

### EXAMPLE 23

A lacrimal canalicular insert is made as described in Example 2, except timolol is used instead of dexamethasone.

### EXAMPLE 24

A lacrimal canalicular insert is made as described in Example 3, except timolol is used instead of dexamethasone.

### EXAMPLE 25

A lacrimal canalicular insert is made as described in Example 1, except memantine is used instead of dexamethasone.

### EXAMPLE 26

A lacrimal canalicular insert is made as described in Example 2, except memantine is used instead of dexamethasone.

### EXAMPLE 27

A lacrimal canalicular insert is made as described in Example 3, except memantine is used instead of dexamethasone.

### EXAMPLE 28

A lacrimal canalicular insert is made as described in Example 1, except a combination of cyclodextrin and prednisolone acetate is used instead of dexamethasone.

### EXAMPLE 29

A lacrimal canalicular insert is made as described in Example 2, except a combination of cyclodextrin and prednisolone acetate is used instead of dexamethasone.

### EXAMPLE 30

A lacrimal canalicular insert is made as described in Example 3, except a combination of cyclodextrin and prednisolone acetate is used instead of dexamethasone.

### EXAMPLES 31-60

Examples 1-30 are repeated except a polylactic acid polymer is used instead of the PLGA copolymer.

### EXAMPLES 61-90

Examples 1-30 are repeated except non-biodegradable ethyl cellulose is used instead of the PLGA copolymer.

### EXAMPLES 91-120

Examples 1-30 are repeated except collagen is used instead of the PLGA copolymer.

In view of the disclosure herein, certain aspects of the present invention can be understood from the following information.

For example, one example of the present biodegradable lacrimal canalicular inserts comprises a biodegradable polymer component and a therapeutic component in a member structured to be placed in a lacrimal canaliculus of an individual and to release the therapeutic component to provide a benefit to the individual.

In one embodiment, the foregoing insert is structured, when placed in a lacrimal canaliculus of an individual, to release the therapeutic component to at least one of an eye, a nasolacrimal system, and a nose of the individual. The insert may be structured so that the therapeutic component is released from the insert for at least about one month after the insert is placed in the lacrimal canaliculus. The biodegradable polymer component of the insert may comprise a synthetic polymer, at least two different biodegradable polymers, or at least one biodegradably copolymer. In certain inserts, the biodegradable polymer component comprises at least one polymer selected from the group consisting of poly lactic acid, poly glycolic acid, poly lactic acid/glycolic acid, derivatives thereof, and mixtures thereof.

In certain embodiments, the therapeutic component of the foregoing insert comprises at least two different therapeutic agents. In some embodiments, the therapeutic component comprises at least one therapeutic agent selected from the group consisting of steroidal anti-inflammatory agents, non-steroidal anti-inflammatory agents, retinoids, prostaglandins, tyrosine kinase inhibitors, adrenoreceptor agonists, adrenoreceptor antagonists, dopaminergic agonists, cholinergic agonists, carbonic anhydrase inhibitors, guanylate cyclase activators, cannabinoids, endothelin, adenosine agonists, antianagiogenic compounds, angiostatic compounds, neuroprotectants, analgesics, antipyretics; antihistamines, antibiotics, beta blockers, anti-neoplastic agents, immunosupressive agents, antiviral agents, antioxidants, and mixtures thereof.

The biodegradable polymer component and the therapeutic component of the foregoing insert may be present in an extrusion molded member. In certain inserts, the member comprises a head portion structured to be placed in proximity to a punctum of an individual, and a body portion structured to be placed in a lacrimal canaliculus of the individual. In addition, some inserts may comprise a body portion that comprises a distal end and a neck located between the distal end and the head portion, wherein the distal end has a greater diameter relative to the diameter of the neck. The distal end of the foregoing insert may comprise a barb, or it may be barbless.

The member of the foregoing inserts may also comprise a structural enhancement component effective in maintaining the strength and physical structure of the member. For example, one of the foregoing inserts may comprise a structural enhancement component that comprises a non-biodegradable polymer mixed with the biodegradable polymer component and the therapeutic component.

In certain inserts, the member further comprises a release modulator effective in modulating release of the therapeutic component from the insert when the insert is placed in a punctal aperture of an individual. Some inserts may comprise a member that contains at least one agent selected from the group consisting of plasticizers, stabilizers, buffers, and salts.

The member of the foregoing inserts may have a peripheral surface, and the insert may further comprise a coating located on the peripheral surface except for portions of the peripheral surface which contact an eye of the individual, the coating being substantially impermeable to the therapeutic component. For example, a coating may comprise a non-biodegradable polymer. The member of the inserts may also comprise a distal end structured to be placed in a lacrimal canaliculus of the individual, and an aperture in the coating provided at the distal end of the member. The aperture may form the distal end of an axial bore extending through the member.

One example of the foregoing inserts comprises a therapeutic component that comprises at least one agent selected from the group consisting of brimonidine, brimonidine salts, and mixtures thereof. In another example, the therapeutic component comprises a combination of (i) brimonidine, salts thereof, and mixtures thereof, and (ii) timolol, salts thereof, and mixtures thereof. In yet another example, the therapeutic component comprises at least one agent selected from the group consisting of bimatoprost, latanoprost, travoprost, unoprostone isopropyl, and salts thereof. In one embodiment, the therapeutic component consists essentially of bimatoprost. In an additional example, the therapeutic component comprises at least one agent selected from the group consisting of cyclosporine, salts thereof, and mixtures thereof. The therapeutic component of another insert may comprise at least one agent selected from the group consisting of predinsolone acetate, salts thereof, and mixtures thereof. Or, the therapeutic component of an insert may comprise a combination of (i) a cyclodextrin, salts thereof, and mixtures thereof, and (ii) prednisolone acetate, salts thereof, and mixtures thereof. The therapeutic component of certain inserts comprises at least one of dexamethasone, salts thereof, and mixtures thereof. Another insert comprises a therapeutic component that includes at least one agent selected from the group consisting of timolol, salts thereof, and mixtures thereof. An additional insert comprises a therapeutic component that includes at least one agent selected from the group consisting memantine, salts thereof, and mixtures thereof. Another insert has a therapeutic component that comprises at least one agent selected from the group consisting of triamcinolone, salts thereof, and mixtures thereof. One insert comprises a therapeutic component that comprises triamcinolone acetate. Another insert comprises a therapeutic component that comprises a non-steroidal antiinflammatory agent. Other inserts may comprise a therapeutic component that comprises at least one agent selected from the group consisting of nepafenac, salts thereof, and mixtures thereof.

In at least one specific embodiment, a biodegradable lacrimal canalicular insert, comprises an extrusion molded member comprising a blend of at least one biodegradable polymer and at least one therapeutic agent. The member may be structured to release the therapeutic agent for an extended period of time. For example, the member may be structured to release the therapeutic agent for at least about one month after placement in a lacrimal canaliculus of an individual. The member may have a peripheral surface, and a non-biodegradable coating circumscribing the peripheral surface. The coating may be located on one or more portions of the peripheral surface other than the portion or portions of the peripheral surface that contact an eye of an individual when the insert is in use. The inserts may also comprise an axial bore extending through the member.

Another specific embodiment of the present invention is a lacrimal canalicular insert, comprising a matrix of a polymeric component and a therapeutic component, wherein the therapeutic component is distributed substantially throughout the matrix. The matrix may be structured in the form of a punctal plug. The polymeric component of the insert may comprise at least one non-biodegradable polymer, or at least one biodegradable polymer. Some of these inserts may comprise a non-biodegradable coating around a major portion of the matrix. The insert may be structured to substantially occlude the lacrimal canaliculus in which it is placed after the therapeutic component has been released therefrom. Certain inserts may comprise a therapeutic component that comprises at least two therapeutic agents. The foregoing insert may comprise a matrix that is an extrusion molded blend of the polymeric component and the therapeutic component. The inserts may be structured to provide release of the therapeutic component from the insert for at least about one month after the insert is placed in a lacrimal canaliculus.

In another specific example, a lacrimal canalicular insert comprises a therapeutic component and a solubilizing enhancing amount of a solubility enhancing component. In some inserts, the amount of the solubility enhancing component is effective in enhancing delivery of the therapeutic component to the posterior of an eye of an individual relative to a substantially identical insert without a solubility enhancing component.

In yet another specific example, a lacrimal canalicular insert comprises a cyclodextrin and at least one therapeutic agent. Certain inserts may also comprise a biodegradable polymer component.

Another aspect of the invention provides a method of producing a lacrimal canalicular insert. One example of such a method comprises forming at least one biodegradable polymer and at least one therapeutic agent into a member structured to be placed in a lacrimal canaliculus of an individual.

The foregoing method may comprise extrusion molding a mixture comprising the biodegradable polymer and the therapeutic agent. The foregoing method may also comprise applying a coating to a peripheral surface of the member. In addition, the method may comprise forming an axial bore in the member.

The present invention also encompasses the use of at least one biodegradable polymer and at least one therapeutic agent in the manufacture of a member structured to be placed in a lacrimal canaliculus of an individual.

A method of treating a condition of a human or animal in accordance with the disclosure herein may comprise placing any one of the foregoing inserts into a lacrimal canaliculus of the human or animal.

The condition treated by the foregoing method may effect at least one of an eye, a nasolacrimal system, and a nose of the human or animal. The insert used in the foregoing method may be effective in releasing the therapeutic component from the insert for at least about one month after being placed in the lacrimal canaliculus.

The foregoing method may also comprise administering at least one other therapeutic agent to the eye of the human or animal. For example, a method may comprise topical administration of at least one other therapeutic agent to the eye of the human or animal.

As one example, a method of treating an ophthalmic condition of an eye comprises administering to an eye of a mammal in need thereof a combination of a therapeutic agent and a cyclodextrin to the eye via a lacrimal canalicular insert, wherein the administering of the combination is effective in treating a disease affecting a posterior portion of the eye. In certain methods, the therapeutic agent is a lipophilic therapeutic agent.

The therapeutic agent used in the foregoing method may comprise at least one of prednisolone, salts thereof, and mixtures thereof. In one method, the therapeutic agent is prednisolone acetate.

The administering step of the foregoing method may be effective in delivering a greater amount of the therapeutic agent to the posterior portion of the eye relative to a substantially identical therapeutic agent topically administered to an eye without cyclodextrin.

Another method of treating an ophthalmic condition of an eye, comprises administering to an eye of a mammal in need thereof, a combination of a therapeutic agent and a solubility enhancing agent to the eye via a lacrimal canalicular insert, wherein the administering of the combination is effective in treating a disease affecting a posterior portion of the eye. For example, the solubility enhancing component may comprise cyclodextrin.

Another method of treating an ophthalmic condition of an eye, comprises administering to an eye of a mammal in need thereof, at least one therapeutic agent via a lacrimal canalicular insert; and administering to the eye of the mammal, an amount of cyclodextrin effective in delivering a therapeutically effective amount of the therapeutic agent to a posterior portion of the eye. For example, the cyclodextrin may be separately administered to the eye. In certain methods, the cyclodextrin is administered in the form of a liquid, an ointment, or a gel. Another aspect of the present invention relates to the use of a biodegradable polymer component and a therapeutic component in the manufacture of a lacrimal canalicular insert or other similar device or member for treating a condition, such as an ophthalmic condition, as described hereinabove.

While this invention has been described with respect to various specific examples and embodiments, it is to be understood that the invention is not limited thereto and that it can be variously practiced within the scope of the following claims.

A number of references, including without limitation patents, patent applications, and patent publications, have been identified herein. Each of these references in its entirety is hereby incorporated by reference.

What is claimed is:

1. A biodegradable lacrimal canalicular insert, comprising:
a biodegradable polymer component and a therapeutic component in a member structured to be placed in a lacrimal canaliculus of an individual and to release the therapeutic component to provide a benefit to the individual.

2. The insert of claim 1 structured, when placed in a lacrimal canaliculus of an individual, to release the therapeutic component to at least one of an eye, a nasolacrimal system, and a nose of the individual.

3. The insert of claim 1, wherein the biodegradable polymer component comprises at least one biodegradable copolymer.

4. The insert of claim 1, wherein the biodegradable polymer component comprises at least one polymer selected from the group consisting of poly lactic acid, poly glycolic acid, poly lactic acid/glycolic acid, derivatives thereof, and mixtures thereof.

5. The insert of claim 1, wherein the therapeutic component comprises at least one therapeutic agent selected from the group consisting of steroidal anti-inflammatory agents, non-steroidal anti-inflammatory agents, retinoids, prostaglandins, tyrosine kinase inhibitors, adrenoreceptor agonists, adrenoreceptor antagonists, dopaminergic agonists, cholinergic agonists, carbonic anhydrase inhibitors, guanylate cyclase activators, cannabinoids, endothelin, adenosine agonists, antianagiogenic compounds, angiostatic compounds, neuroprotectants, analgesics, antipyretics; antihistamines, antibiotics, beta blockers, anti-neoplastic agents, immunosupressive agents, antiviral agents, antioxidants, and mixtures thereof.

6. The insert of claim 1, wherein the member comprises a head portion structured to be placed in proximity to a punctum of an individual, and a body portion structured to be placed in a lacrimal canaliculus of the individual.

7. The insert of claim 6, wherein the body portion comprises a distal end and a neck located between the distal end and the head portion, wherein the distal end has a greater diameter relative to the diameter of the neck.

8. The insert of claim 1, wherein the member has a peripheral surface, and wherein the insert further comprises a coating located on the peripheral surface except for portions of the peripheral surface which contact an eye of the individual, the coating being substantially impermeable to the therapeutic component.

9. The insert of claim 8, wherein the member comprises a distal end structured to be placed in a lacrimal canaliculus of the individual, and an aperture in the coating provided at the distal end of the member.

10. The insert of claim 1, which is an extrusion molded member comprising a blend of at least one biodegradable polymer and at least one therapeutic agent.

11. The insert of claim 1, wherein the therapeutic component comprises a combination of (i) brimonidine, salts thereof, and mixtures thereof, and (ii) timolol, salts thereof, and mixtures thereof.

12. The insert of claim 1, wherein the therapeutic component comprises at least one agent selected from the group consisting of bimatoprost, latanoprost, travoprost, unoprostone isopropyl, and salts thereof.

13. The insert of claim 1, wherein the therapeutic component comprises at least one agent selected from the group consisting of cyclosporine, salts thereof, and mixtures thereof.

14. The insert of claim 1, wherein the therapeutic component comprises at least one agent selected from the group consisting of predinsolone acetate, salts thereof, and mixtures thereof.

15. The insert of claim 1, wherein the therapeutic component comprises at least one agent selected from the group consisting memantine, salts thereof, and mixtures thereof.

16. The insert of claim 1, wherein the therapeutic component comprises at least one agent selected from the group consisting of triamcinolone, salts thereof, and mixtures thereof.

17. The insert of claim 1, wherein the therapeutic component comprises triamcinolone acetate.

18. The insert of claim 1, wherein the therapeutic component comprises a non-steroidal antiinflammatory agent.

19. A method of producing a lacrimal canalicular insert, comprising:
forming at least one biodegradable polymer and at least one therapeutic agent into a member structured to be placed in a lacrimal canaliculus of an individual.

20. A method of treating a condition of a human or animal, comprising placing the insert of claim 1 into a lacrimal canaliculus of the human or animal to treat the condition of at least one of an eye, a nasolacrimal system, and a nose of the human or animal.

21. Use of at least one biodegradable polymer and at least one therapeutic agent in the manufacture of a biodegradable lacrimal canalicular insert.

22. Use of at least one biodegradable polymer and at least one therapeutic agent in the manufacture of a lacrimal canalicular insert for treating an ophthalmic condition.
